(19) United States
(12) Patent Application Publication
(10) Pub. No.: US 2016/0374743 A1
(43) Pub. Date: Dec. 29, 2016

(54) NEUROMODULATION CATHETERS AND RELATED DEVICES, SYSTEMS, AND METHODS

(71) Applicant: MEDTRONIC ARDIAN LUXEMBOURG S.A.R.L., Luxembourg (LU)

(72) Inventors: Rudy BEASLEY, Santa Rosa, CA (US); Don TRAN, Santa Rosa, CA (US); William BERTHIAUME, Santa Rosa, CA (US); Justin GOSHGARIAN, Santa Rosa, CA (US); Charlie CONSIDINE, Santa Rosa, CA (US); Susheel DESHMUKH, Santa Rosa, CA (US); Rajeshkumar DHAMODHARASAMY, Santa Rosa, CA (US); Sina SOM, Santa Rosa, CA (US); Sukyoung SHIN, Santa Rosa, CA (US); Jaime RIOS, Santa Rosa, CA (US)

(21) Appl. No.: 14/783,231

(22) PCT Filed: Mar. 20, 2015
(86) PCT No.: PCT/US2015/021835

§ 371 Date: Oct. 8, 2015

Related U.S. Application Data
(60) Provisional application No. 61/968,310, filed on Mar. 20, 2014.

(51) Int. Cl. A61B 18/04; A61M 39/10; A61M 25/00; A61N 1/36; A61N 1/40

(52) U.S. Cl. CPC A61B 18/04; A61N 1/36117; A61N 1/403; A61M 25/0009; A61M 39/1011; A61B 2018/00577

(57) ABSTRACT

A neuromodulation catheter in accordance with a particular embodiment includes an elongate shaft and a neuromodulation element operably connected to the shaft. The shaft includes a proximal hypotube segment at its proximal end portion and a jacket disposed around at least a portion of an outer surface of the hypotube segment. The jacket may be made at least partially of a polymer blend including polyether block amide and polysiloxane. The neuromodulation element includes a distal hypotube segment and a tubular jacket disposed around at least a portion of an outer surface of the distal hypotube segment. The jacket has reduced-diameter segments spaced apart along its longitudinal axis. The neuromodulation element further includes band electrodes respectively seated in the reduced-diameter segments and respectively forming closed loops extending circumferentially around the jacket.

### RELATED APPLICATIONS

This application claims the benefit of U.S. Provisional Patent Application 61/968,310, filed Mar. 20, 2014, the disclosure of which is herein incorporated by reference in its entirety.

### TECHNICAL FIELD

The present technology is related to catheters, such as neuromodulation catheters.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic activation of the SNS, however, is a common maladaptive response that can drive the progression of many disease states. Excessive activation of the renal SNS, in particular, has been identified experimentally and in humans as a likely contributor to the complex pathophysiologies of hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function are considerably stimulated in disease states characterized by heightened sympathetic tone. For example, reduced renal blood flow and glomerular filtration rate as a result of renal sympathetic efferent stimulation is likely a cornerstone of the loss of renal function in cardio-renal syndrome (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter renal sympathetic mediated sodium and water retention). These pharmacologic strategies, however, have significant limitations including limited efficacy, compliance issues, side effects, and others.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. For ease of reference, throughout this disclosure identical reference numbers may be used to identify identical or at least generally similar or analogous components or features.

FIG. 1 is a partially schematic perspective view illustrating a therapeutic system configured in accordance with an embodiment of the present technology.

FIG. 2 is an exploded profile view of a catheter of the system shown in FIG. 1.

FIGS. 3 and 4 are enlarged exploded profile views of respective portions of the catheter taken at respective locations designated in FIG. 2.

FIG. 5 is a further enlarged cross-sectional view of an intermediate tube of a shaft of the catheter taken along a line 5-5 designated in FIG. 4.

FIG. 6 is an enlarged exploded profile view of a portion of the catheter taken at a location designated in FIG. 2.

FIG. 7 is a profile view of a proximal hypotube segment of the shaft.

FIG. 8 is a cross-sectional view of the proximal hypotube segment and a proximal jacket of the shaft taken along a line 8-8 designated in FIG. 7.

FIG. 9 is an enlarged profile view of a portion of the proximal hypotube segment and the proximal jacket taken at a location designated in FIG. 7.

FIG. 10 is a profile view of band electrodes and a distal jacket of a neuromodulation element of the catheter.

FIGS. 11 and 12 are, respectively, a perspective view and a profile view of the distal jacket.

FIG. 13 is an enlarged profile view of a portion of the distal jacket taken at a location designated in FIG. 12.

FIG. 14 is a cross-sectional f the distal jacket taken along a line 14-14 designated in FIG. 12.

FIGS. 15, 16 and 17 are enlarged cross-sectional views respectively illustrating a portion of the distal jacket designated in FIG. 14 before, during, and after installation of one of the band electrodes in accordance with an embodiment of the present technology.

FIG. 18 is a flow chart illustrating a method for making the neuromodulation element in accordance with an embodiment of the present technology.

FIG. 19 is an exploded perspective view of a shell of a handle of the catheter.

FIGS. 20, 21 and 22 are, respectively, a plan view, an end profile view, and a side profile view of a first shell segment of the shell.

FIG. 23 is a cross-sectional view of the first shell segment taken along a line 23-23 designated in FIG. 22.

FIGS. 24, 25, and 26 are, respectively, a plan view, an end profile view, and a side profile view of a second shell segment of the shell.

FIG. 27 is a cross-sectional view of the second shell segment taken along a line 27-27 designated in FIG. 26.

FIGS. 28 and 29 are, respectively, an end profile view and a plan view of a coupler of the catheter and the proximal hypotube segment.

FIG. 30 is a cross-sectional view of the coupler and the proximal hypotube segment taken along a line 30-30 designated in FIG. 29.

FIG. 31 is a perspective view of the coupler, the first shell segment, and the proximal hypotube segment.

FIGS. 32 and 33 are, respectively, a perspective view and a side profile view of a distal strain-relief element of the handle.

FIG. 34 is a cross-sectional view of the distal strain-relief element taken along a line 34-34 designated in FIG. 33.

### DETAILED DESCRIPTION

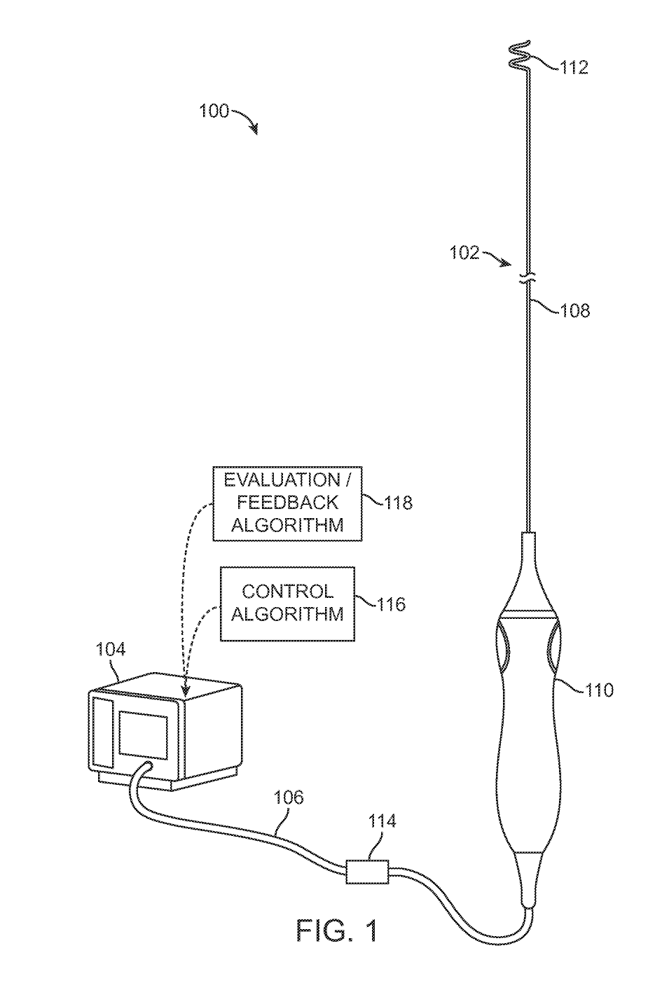

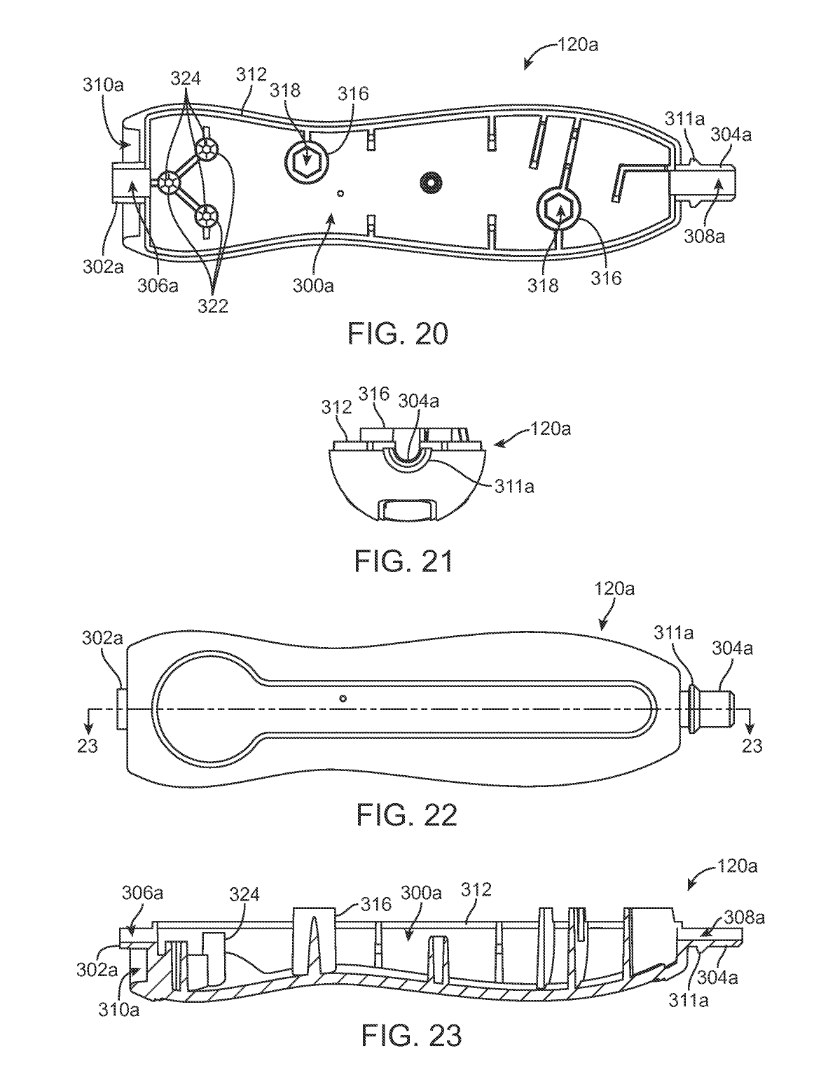

Specific details of systems, devices, and methods in accordance with several embodiments of the present technology are disclosed herein with reference to FIGS. 1-34. Although the systems, devices, and methods may be disclosed herein primarily or entirely with respect to intravascular renal neuromodulation, other applications in addition to those disclosed herein are within the scope of the present technology. For example, systems, devices, and methods in accordance with at least some embodiments of the present technology may be useful for neuromodulation within one or more non-vessel body lumen, for extravascular neuromodulation, for non-renal neuromodulation, and/or for use in therapies other than neuromodulation. Furthermore, it should understood, in general, that other systems, devices, and methods in addition to those disclosed herein are within the scope of the present technology. For example, systems, devices, and methods in accordance with embodiments of the present technology can have different and/or additional configurations, components, and procedures than those disclosed herein. Moreover, a person of ordinary skill in the art will understand that systems, devices, and methods in accordance with embodiments of the present technology can be without one or more of the configurations, components, and/or procedures disclosed herein without necessarily deviating from the present technology.

As used herein, the terms “distal” and “proximal” define a position or direction with respect to a clinician or a clinician's control device (e.g., a handle of a catheter). The terms, “distal” and “distally” refer to a position distant from or in a direction away from a clinician or a clinician's control device. The terms “proximal” and “proximally” refer to a position near or in a direction toward a clinician or a clinician's control device. The headings provided herein are for convenience only and should not be construed as limiting the subject matter disclosed.

### Selected Examples of Neuromodulation Catheters and Technology

FIG. 1 is a partially schematic perspective view illustrating a therapeutic system 100 configured in accordance with an embodiment of the present technology. The system 100 can include a neuromodulation catheter 102, a console 104, and a cable 106 extending therebetween. The catheter 102 can include an elongate shaft 108, a handle 110 operably connected to the shaft 108 via a proximal end portion of the shaft 108, and a neuromodulation element 112 operably connected to the shaft 108 via a distal end portion of the shaft 108. The shaft 108 can be configured to locate the neuromodulation element 112 at a treatment location within or otherwise proximate to a body lumen (e.g., a blood vessel, a duct, an airway, or another naturally occurring lumen within the human body). In at least some embodiments, the shaft 108 is configured to locate the neuromodulation element 112 at an intraluminal (e.g., intravascular) location. The neuromodulation element 112 can be configured to provide or support a neuromodulation treatment at the treatment location. The shaft 108 and the neuromodulation element 112 can be 2, 3, 4, 5, 6, or 7 French or other suitable sizes.

Intraluminal delivery of the catheter 102 can include percutaneously inserting a guide wire (not shown) into a body lumen of a patient and moving the shaft 108 and the neuromodulation element 112 along the guide wire until the neuromodulation element 112 reaches a suitable treatment location. Alternatively, the catheter 102 can be a steerable or non-steerable device configured for use without a guide wire. As another alternative, the catheter 102 can be configured for use with a guide catheter or sheath (not shown). In the illustrated embodiment, the console 104 is configured to control, monitor, supply, and/or otherwise support operation of the catheter 102. In other embodiments, the catheter 102 can be self-contained or otherwise configured for operation independent of the console 104. When present, the console 104 can be configured to generate a selected form and/or magnitude of energy for delivery to tissue at a treatment location via the neuromodulation element 112. For example, the console 104 can be configured to generate radio frequency (RF) energy (e.g., monopolar and/or bipolar RE energy) and/or another suitable type of energy for delivery to tissue at a treatment location via electrodes (not shown in FIG. 1) of the neuromodulation element 112. Along the cable 106 or at another suitable location within the system 100, the system 100 can include a control device 114 (shown schematically) configured to initiate, terminate, and/or adjust operation of one or more components of the catheter 102 directly and/or via the console 104. The console 104 can be configured to execute an automated control algorithm 116 and/or to receive control instructions from an operator. Similarly, the console 104 can be configured to provide feedback to an operator before, during, and/or after a treatment procedure via an evaluation/feedback algorithm 118.

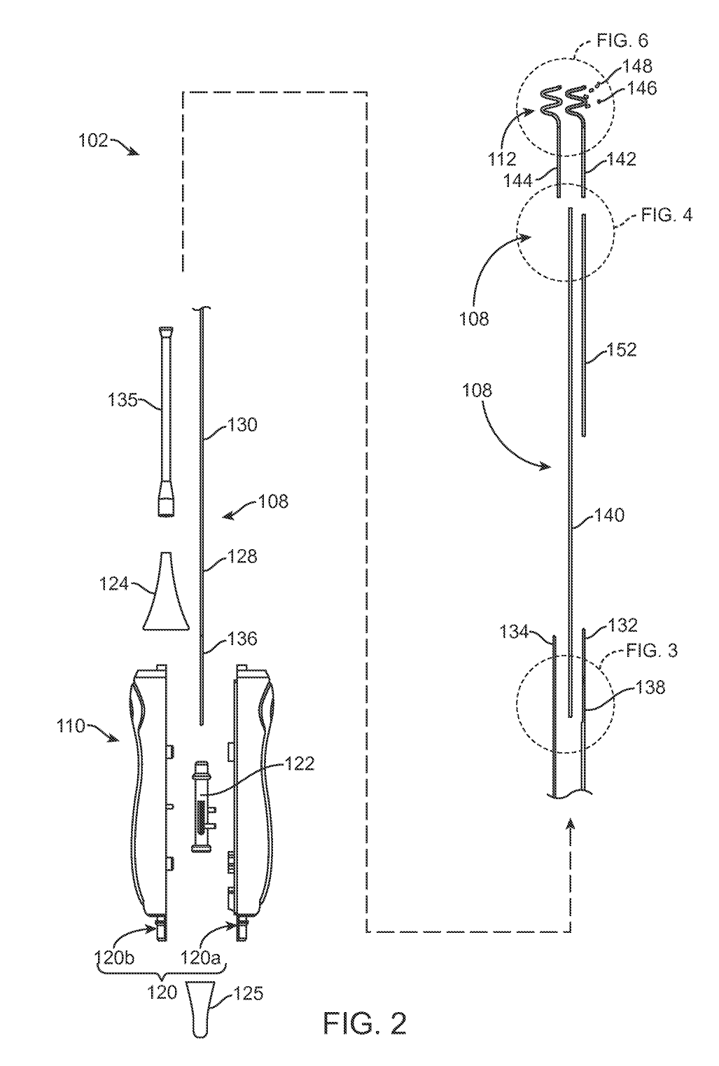

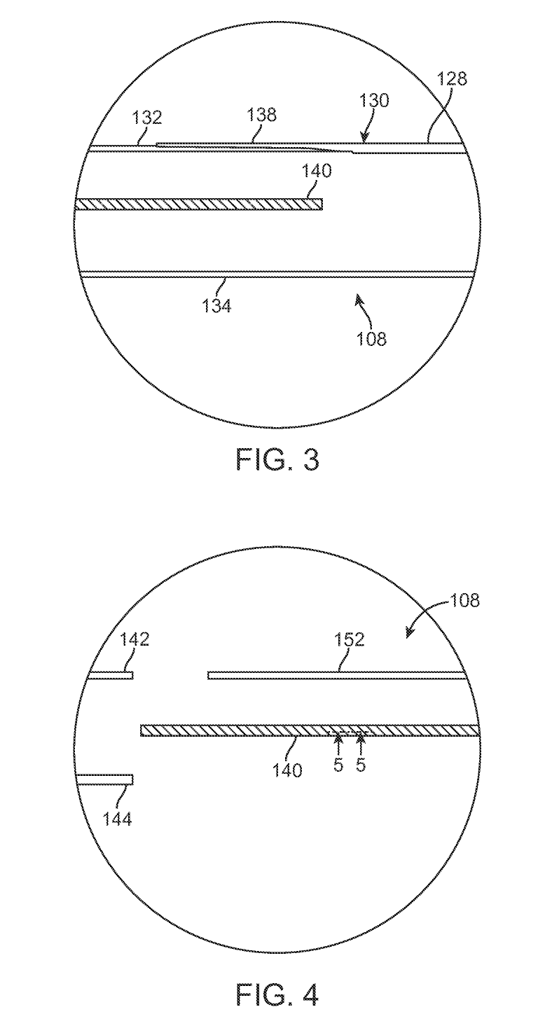

FIG. 2 is an exploded profile view of the catheter 102. FIGS. 3, 4 and 6 are enlarged exploded profile views of respective portions of the catheter 102 taken at respective locations designated in FIG. 2. With reference to FIGS. 1-4 and 6 together, the handle 110 can include a shell 120 having a first shell segment 120a and a second shell segment 120b releasably connectable to one another. The shell 120 can define a cavity (not shown in FIG. 2) between the first and second shell segments 120a, 120b. Disposed at least partially within the cavity, the catheter 102 can include a coupler 122 configured to releasably connect the shaft 108 to the handle 110. In some embodiments, the handle 110 is configured for multiple uses whereas the shaft 108, the neuromodulation element 112, and the coupler 122 are disposable after fewer uses, such as one or more uses on a single patient. In other embodiments, the handle 110, the shaft 108, the neuromodulation element 112, and the coupler 122 can have other suitable usage protocols.

As shown in FIG. 2, the handle 110 can include a distally tapered distal strain-relief element 124 and a proximally tapered proximal strain-relief element 125 releasably connected to distal and proximal ends, respectively, of the shell 120. When a portion of the catheter 102 is disposable, it can be useful for the handle 110 to disassemble readily to allow the disposable portion to be replaced. When operably connected to the shell 120, the distal and proximal strain-relief elements 124, 125 can releasably inhibit separation of the first and second shell segments 120a, 120b. Conversely, separating the distal and proximal strain-relief elements 124, 125 from the shell 120, such as by advancing the distal strain-relief element 124 away from the shell 120 along the shaft 108 and advancing the proximal strain-relief element 125 away from the shell 120 along the cable 106 (FIG. 1), can allow the first and second shell segments 120a, 120b to separate.

The shaft 108 can include an assembly of parallel elongate tubular segments. For example, the shaft 108 can include a proximal hypotube segment 128, a proximal jacket 130, a first electrically insulative tube 132, and a guide-wire tube 134. Slidably positioned over the shaft 108, the catheter 102 can include a loading tool 135 configured to facilitate loading the catheter 102 onto a guide wire. The first electrically insulative tube 132 and the guide-wire tube 134 can be disposed side-by-side within the proximal hypotube segment 128. The first electrically insulative tube 132 can be configured to carry electrical leads (not shown) and to electrically insulate the electrical leads from the proximal hypotube segment 128. The guide-wire tube 134 can be configured to carry the guide wire. The proximal jacket 130 can be disposed around at least a portion of an outer surface of the proximal hypotube segment 128. The proximal hypotube segment 128 can include a stern 136 at its proximal end and a skive 138 at its distal end.

The first electrically insulative tube 132 and the guide-wire tube 134 can extend distally beyond the skive 138. The shaft 108 can include an intermediate tube 140 beginning proximally at a region of the shaft 108 at which the first electrically insulative tube 132 and the guide-wire tube 134 distally emerge from the proximal hypotube segment 128. The intermediate tube 140 can be more flexible than the proximal hypotube segment 128. At the region of the shaft 108 at which the first electrically insulative tube 132 and the guide-wire tube 134 distally emerge from the proximal hypotube segment 128, the intermediate tube 140 can be coaxially aligned with the proximal hypotube segment 128 so as to receive the first electrically insulative tube 132 and the guide-wire tube 134. From this region, the intermediate tube 140 can extend distally to the distal end portion of the shaft 108. The first electrically insulative tube 132 can distally terminate within the intermediate tube 140. In contrast, the guide-wire tube 134 can extend through the entire length of the intermediate tube 140. At a distal end of the intermediate tube 140, the shaft 108 can be operably connected to the neuromodulation element 112.

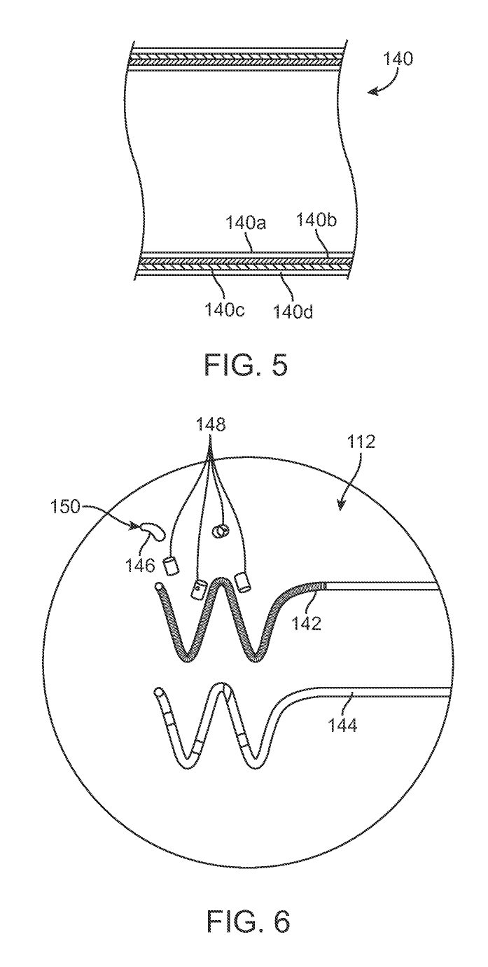

FIG. 5 is a further enlarged cross-sectional view of the intermediate tube 140 taken along a line 5-5 designated in FIG. 4. Arranged from innermost to outermost, the intermediate tube 140 can include an inner polymer layer 140a, a metal braid 140b, a first outer polymer layer 140c, and a second outer polymer layer 140d. In some embodiments, the inner polymer layer 140a is made of polyimide, the metal braid 140b is made of stainless steel, the first outer polymer layer 140c is made of coated (e.g., triple-coated) polyimide, and the second outer polymer layer 140d is made of polyether block amide (e.g., PEBAX®). In these and other embodiments, the thicknesses of the inner polymer layer 140a and the second outer polymer layer 140d can be about 0.006 inch and about 0.00125 inch, respectively. In other embodiments, components of the intermediate tube 140 can have other suitable compositions and/or dimensions.

In at least some embodiments, some or all of the intermediate tube 140 is film-cast. For example, the first outer polymer layer 140c can be disposed onto the metal braid 140b as a series of thin films. This can allow the thickness of the first outer polymer layer 140c to be precisely controlled. Accordingly, the first outer polymer layer 140c can be just thick enough to prevent the ends of the metal braid 140b from becoming exposed or otherwise damaged when thermally bonding the intermediate tube 140 to the proximal and distal hypotube segments 128, 142, respectively, but not so thick as to cause the intermediate tube 140 to become excessively stiff. This can reduce or eliminate the need to locally reinforce the ends of the intermediate tube 140 or to splice coupling components onto the ends of the intermediate tube 140 to facilitate bonding the intermediate tube 140 to the proximal and distal hypotube segments 128, 142.

The neuromodulation element 112 can include a distal hypotube segment 142 coupled to the distal end of the intermediate tube 140. The neuromodulation element 112 can further include a distal jacket 144 disposed around at least a portion of an outer surface of the distal hypotube segment 142. The neuromodulation element 112 can still further include band electrodes 146 disposed outside the distal jacket 144 at spaced-apart positions along a longitudinal axis of the distal jacket 144. At a distal end of the distal hypotube segment 142, the neuromodulation element 112 can include a distally tapering a traumatic tip 148 having a distal opening 150. The guide-wire tube 134 can extend through the distal hypotube segment 142 to the distal opening 150. Electrical leads can extend through the distal hypotube segment 142 to the band electrodes 146, respectively.

In FIGS. 2 and 6, the neuromodulation element 112 is shown in a radially expanded deployed state. The neuromodulation element 112 can be movable from a low-profile delivery state to the radially expanded deployed state. When the neuromodulation element 112 is in the radially expanded deployed state, the distal hypotube segment 142 can have a shape that is more helical (i.e., spiral) than its shape when the neuromodulation element 112 is in the low-profile delivery state. In at least some cases, the distal hypotube segment 142 has the more helical shape when at rest and is configured to be forced into the less helical shape by an external sheath (not shown).

The distal hypotube segment 142 can be made at least partially of nickel titanium, stainless steel, or another suitable material well suited for resiliently moving between the more helical and less helical shapes. In at least some cases, the material of the distal hypotube segment 142 is electrically conductive. Accordingly, the neuromodulation element 112 can include a second electrically insulative tube 152 disposed around an outer surface of the distal hypotube segment 142 so as to electrically separate the band electrodes 146 from the distal hypotube segment 142. In some embodiments, the first and second electrically insulative tubes 132, 152 are made at least partially (e.g., predominantly or entirely) of polyimide and polyether block amide, respectively. In other embodiments, the first and second electrically insulative tubes 132, 152 can be made of other suitable materials.

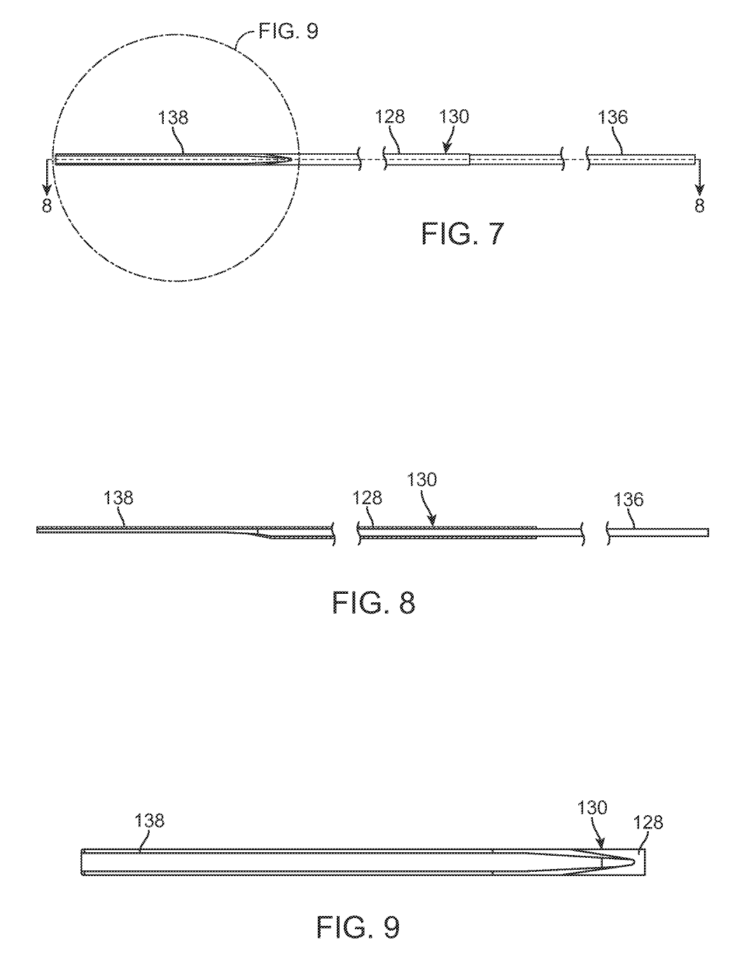

FIG. 7 is a profile view of the proximal hypotube segment 128 and the proximal jacket 130. FIG. 8 is a cross-sectional profile view of the proximal hypotube segment 128 and the proximal jacket 130 taken along a line 8-8 designated in FIG. 7. FIG. 9 is an enlarged profile view of a portion of the proximal hypotube segment 128 and the proximal jacket 130 taken at a location designated in FIG. 7. As shown in FIGS. 7 and 8, the proximal jacket 130 can be absent from the outer surface of the proximal hypotube segment 128 at the stem 136. This can be useful, for example, to facilitate connecting the proximal hypotube segment 128 to the coupler 122. In contrast, the proximal jacket 130 can be disposed on at least a portion of the outer surface of the proximal hypotube segment 128 at the skive 138. This can be useful, for example, to reduce or eliminate the possibility of kinking and/or to otherwise prevent damage to electrical leads extending through the shaft 108 during use of the catheter 102.

The proximal hypotube segment 128 can have features that enhance its flexibility,such as to facilitate delivery of the catheter 102 via a transradial approach or another relatively long and/or tortuous percutaneous transluminal approach. Several examples of such features are described in U.S. patent application Ser. No. 14/060,573, filed Oct. 22, 2013, which is incorporated herein by reference in its entirety and included with the present application as Appendix 1. In some embodiments, the proximal hypotube segment 128 is made at least partially (e.g., predominantly or entirely) of nickel titanium. Furthermore, all or a suitable portions of the proximal hypotube segment 128 can be heat treated or otherwise formed to have a desirable shape memory transformation temperature range and/or Af temperature, such as a shape memory transformation temperature range from 5° C. to 15° C. and/or an Af temperature within this range. This can facilitate maintaining nickel titanium in the relatively flexible martensite phase while at about 37° C. (i.e., body temperature). In other embodiments, the proximal hypotube segment 128 can have other suitable compositions and/or properties.

The proximal jacket 130 can be made at least partially (e.g., predominantly or entirely) of a polymer blend, such as a polymer blend including polyether block amide and polysiloxane. In some embodiments, the polymer blend includes polysiloxane at greater than 15% by weight, such as greater than 20% by weight or greater than 30% by weight. For example, the polymer blend can include polysiloxane at from 20% to 40% by weight. In a particular embodiment, the polymer blend includes polysiloxane at about 20% by weight and polyether block amide at about 80% by weight. In another embodiment, the polymer blend includes polysiloxane at about 40% by weight and polyether block amide at about 60% by weight. Polymer blends rich in polysiloxane (e.g., having concentrations of polysiloxane greater than 30%) are conventionally thought to be unsuitable for use in catheters, perhaps due to a conventionally observed inverse relationship between polysiloxane concentration and strength. Unexpectedly, polymer blends rich in polysiloxane are expected to outperform polymer blends having lower concentrations of polysiloxane in at least some respects when used in the proximal jacket 130. For example, a relatively high polysiloxane concentration may increase the lubricity to the proximal jacket 130, which may, in turn, reduce friction between the proximal jacket 130 and a delivery sheath during use of the catheter 102. This reduction in friction may outweigh and/or offset any detrimental effect of the relatively high polysiloxane concentration on the strength of the proximal jacket 130. In some cases, polymer blends selected in accordance with embodiments of the present technology may allow the proximal jacket 130 to have sufficient lubricity for use without an outer coating. In other cases, an outer coating may be used. Furthermore, the proximal jacket 130 can have other suitable compositions and/or properties than those described above.

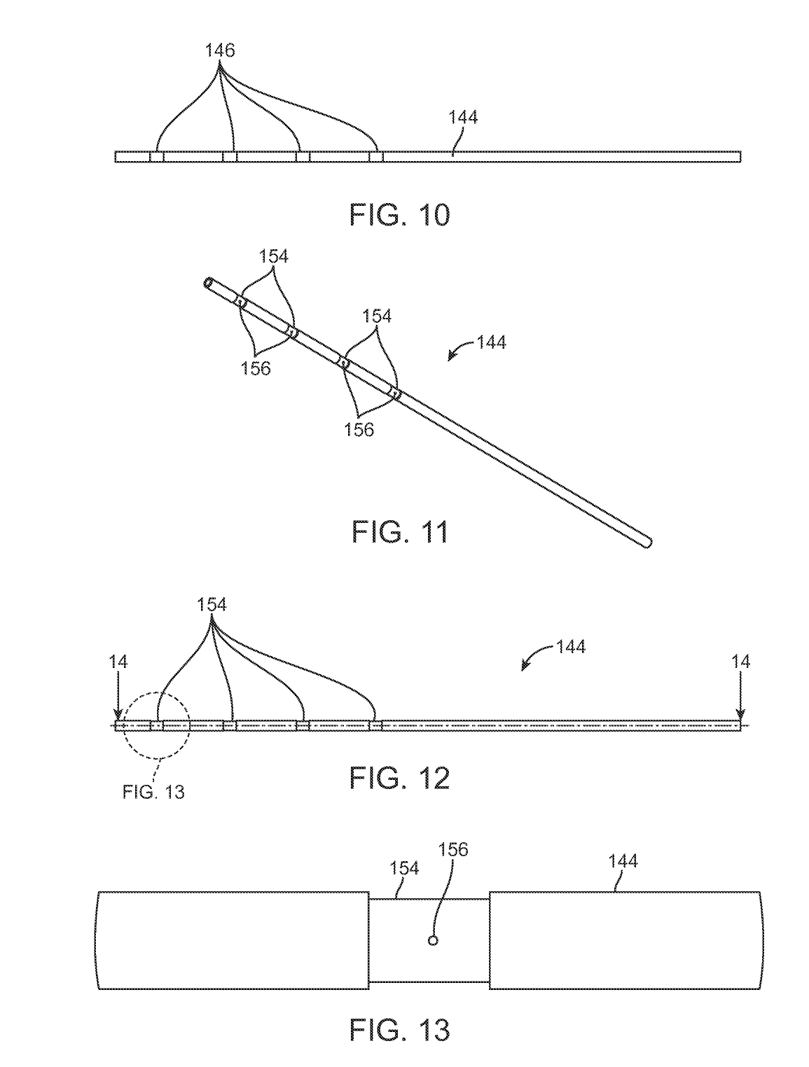

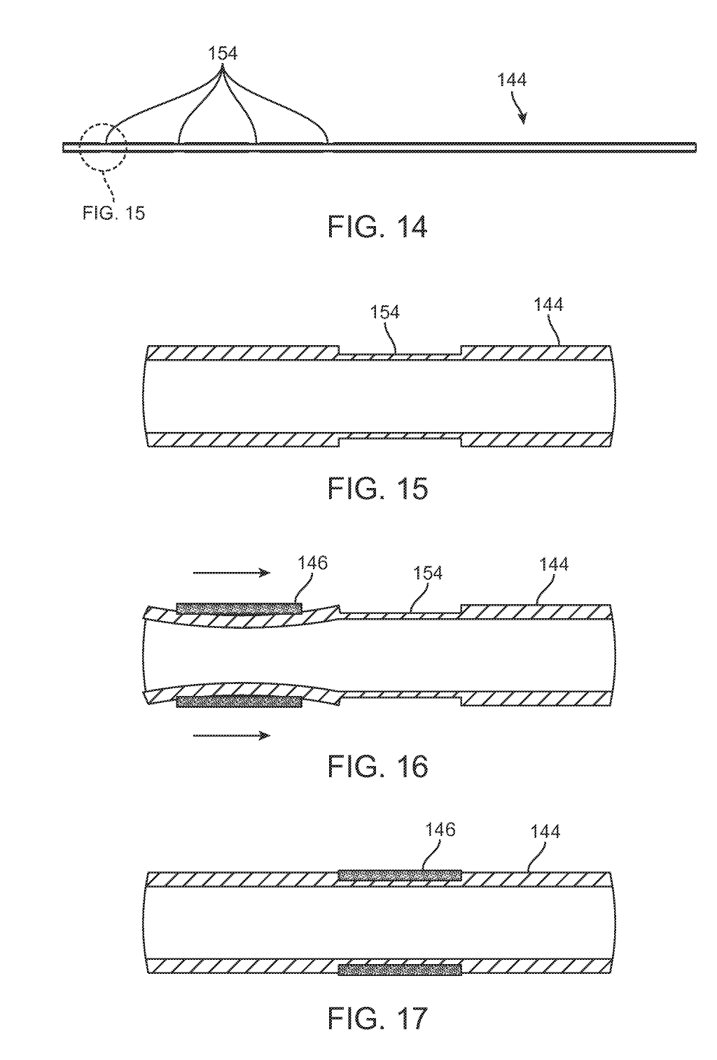

FIG. 10 is a profile view of the band electrodes 146 and the distal jacket 144. FIGS. 11 and 12 are, respectively, a perspective view and a profile view of the distal jacket 144. FIG. 13 is an enlarged profile view of a portion of the distal jacket 144 taken at a location designated in FIG. 12. FIG. 14 is a cross-sectional view of the distal jacket 144 taken along a line 14-14 designated in FIG. 12. With reference to FIGS. 10-14 together, the distal jacket 144 can be tubular and configured to be disposed around at least a portion of an outer surface of the distal hypotube segment 142 (FIGS. 2 and 6). The distal jacket 144 can include reduced-diameter segments 154 extending through its outer surface. The band electrodes 146 can be respectively seated in the reduced-diameter segments 154.

The reduced-diameter segments 154 can be insets, pockets, grooves, or other suitable features configured to respectively seat the band electrodes 146. In the illustrated embodiment, the distal jacket 144 includes exactly four reduced-diameter segments 154 spaced apart along its longitudinal axis. Alternatively, the distal jacket 144 can include exactly one, two, three, five, six or a greater number of reduced-diameter segments 154. The reduced-diameter segments 154 may be spaced apart at equal distances or at different distances. The distal jacket 144 can include openings 156 respectively positioned at the reduced-diameter segments 154. Electrical leads can extend from respective reduced-diameter segments 154, through respective openings 156, through a lumen of the distal hypotube segment 142 (FIGS. 2 and 6), through the intermediate tube 140, and through the proximal hypotube segment 128 to the handle 110. In this way, the electrical leads can respectively connect the band electrodes 146 to proximal components of the catheter 102.

FIGS. 15-17 are enlarged cross-sectional views respectively illustrating a portion of the distal jacket 144 before, during, and after installation of one of the band electrodes 146 in accordance with an embodiment of the present technology. As illustrated in FIGS. 15-17, the portion of the distal jacket 144 can include one of the reduced-diameter segments 154. In FIG. 15, the portion of the distal jacket 144 is illustrated without the band electrode 146 corresponding to the reduced-diameter segment 154. In FIG. 16, the portion of the distal jacket 144 is illustrated resiliently deformed inwardly as the band electrode 146 is moved toward the reduced-diameter segment 154. In FIG. 17, the portion of the distal jacket 144 is illustrated with the band electrode 146 seated in the reduced-diameter segment 154. With reference to FIGS. 10-17 together, the band electrodes 146 can respectively form closed loops extending circumferentially around the distal jacket 144. In at least some cases, a minimum inner diameter of the individual band electrodes 146 is smaller than a maximum outer diameter of distal jacket 144 between the reduced-diameter segments 154. To facilitate assembly, the distal jacket 144, between the reduced-diameter segments 154, can be resilient in response to peristaltic deflection of a magnitude corresponding to a difference between the maximum outer diameter of the distal jacket 144 between the reduced-diameter segments 154 and the minimum inner diameter of the individual band electrodes 146. Suitable materials for the distal jacket 144 include polymer blends including polyurethane and polysiloxane, among others.

A maximum outer diameter of the individual band electrodes 146 and the maximum outer diameter of the distal jacket 144 between the reduced-diameter segments 154 can be at least generally equal (e.g., within 5%, 3%, or 2% of one another). Thus, once the band electrodes 146 are respectively seated in the reduced-diameter segments 154, outer surfaces of the band electrodes 146 and the distal jacket 144 between the reduced-diameter segments 154 can be at least generally flush. This can be useful, for example, to reduce or eliminate potentially problematic ridges (e.g., circumferential steps) at distal and proximal ends of the individual band electrodes 146. This, in turn, can reduce or eliminate the need for fillets (e.g., adhesive fillets, such as glue fillets) at the distal and proximal ends of the individual band electrodes 146. In at least some embodiments, the distal jacket 144 and the band electrodes 146 are bonded to one another without any exposed adhesive. For example, an adhesive (not shown) can be disposed between the band electrodes 146 and the distal jacket 144 at the reduced-diameter segments 154.

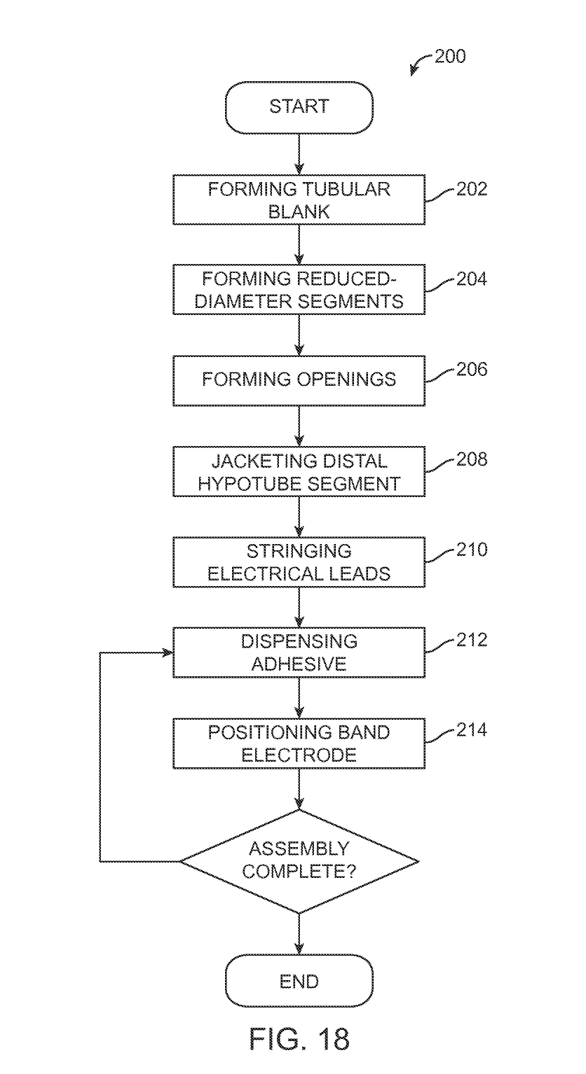

FIG. 18 is a flow chart illustrating a method 200 for making the neuromodulation element 112 in accordance with an embodiment of the present technology. With reference to FIGS. 10-18 together, the method 200 can begin with forming the distal jacket 144. This can include forming a tubular blank (block 202) (e.g., by extrusion) and then using a subtractive process (e.g., by laser ablation) to remove portions of the blank and thereby form the reduced-diameter segments 154 (block 204). The same or a different subtractive process can be used to form the openings 156 (block 206). Alternatively, the distal jacket 144 can be formed by injection molding or another suitable technique that allows the reduced-diameter segments 154 and/or the openings 156 to be formed without the need for a subtractive process. When a subtractive process is used to form the reduced-diameter segments 154, the subtractive process can be precisely controlled so as to leave an innermost portion of a wall of the distal jacket 144 intact at the reduced-diameter segments 154. Laser ablation is one example of a suitable subtractive process for forming the reduced-diameter segments 154. Laser ablation can include loading the blank onto a mandrel and then rotating the blank and the mandrel relative to an ablative laser (or rotating the ablative laser relative to the blank and the mandrel) under computerized control. The mandrel can conductively cool the innermost portion of the wall of the distal jacket 144 so as to prevent this portion of the wall from reaching ablative temperatures at the reduced-diameter segments 154. Other techniques for forming the reduced-diameter segments 154 are also possible.

The method 200 can further include jacketing the distal hypotube segment 142 (block 208), such as by positioning the distal jacket 144 and the distal hypotube segment 142 relative to one another so that the distal jacket 144 is disposed around at least a portion of an outer surface of the distal hypotube segment 142. In at least some embodiments, the form and/or other aspects of the distal jacket 144 may allow the distal jacket 144 to be disposed around at least a portion of the outer surface of the distal hypotube segment 142 without swaging the distal jacket 144. When the distal hypotube segment 142 is positioned within the distal jacket 144, the method 200 can include respectively stringing electrical leads (block 210) from the reduced-diameter segments 154 through a lumen of the distal hypotube segment 142. Next, the method 200 can include dispensing an adhesive (block 212) onto the distal jacket 144 at the reduced-diameter segments 154. Then, the method 200 can include positioning the band electrodes 146 (block 214) at respective reduced-diameter segments 154. As discussed above with reference to FIGS. 15-17, positioning one of the band electrodes 146 can include resiliently deforming the distal jacket 144 inwardly while passing (e,g., threading or otherwise advancing) the distal jacket 144 through a channel of the band electrode 146 so as to move the band electrode 146 toward a longitudinal position at which the band electrode 146 is aligned with the reduced-diameter segment 154. The same process can be used to install the remaining band electrodes 146 in order from proximal to distal.

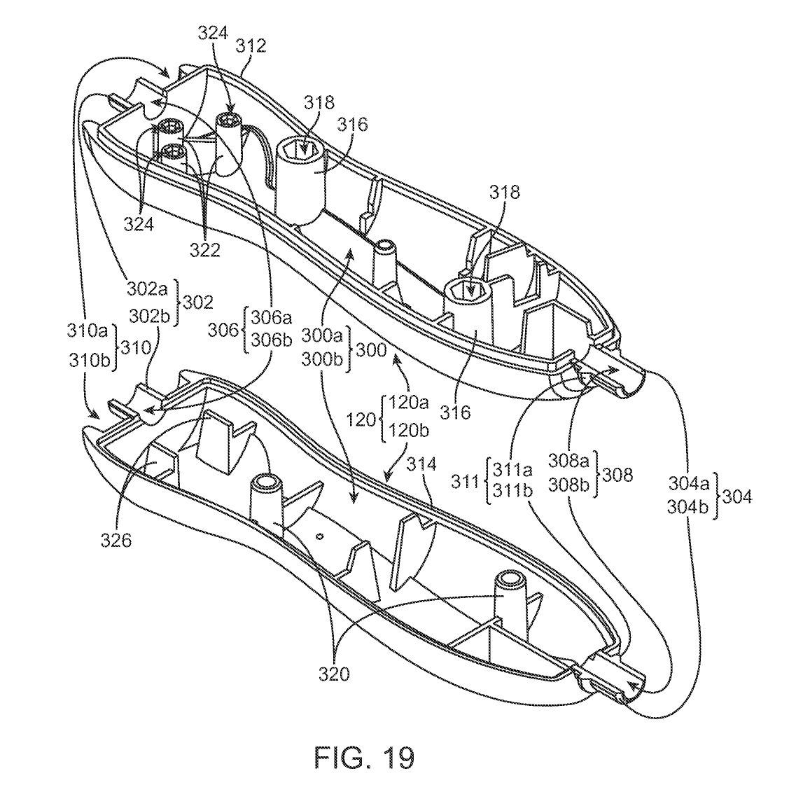

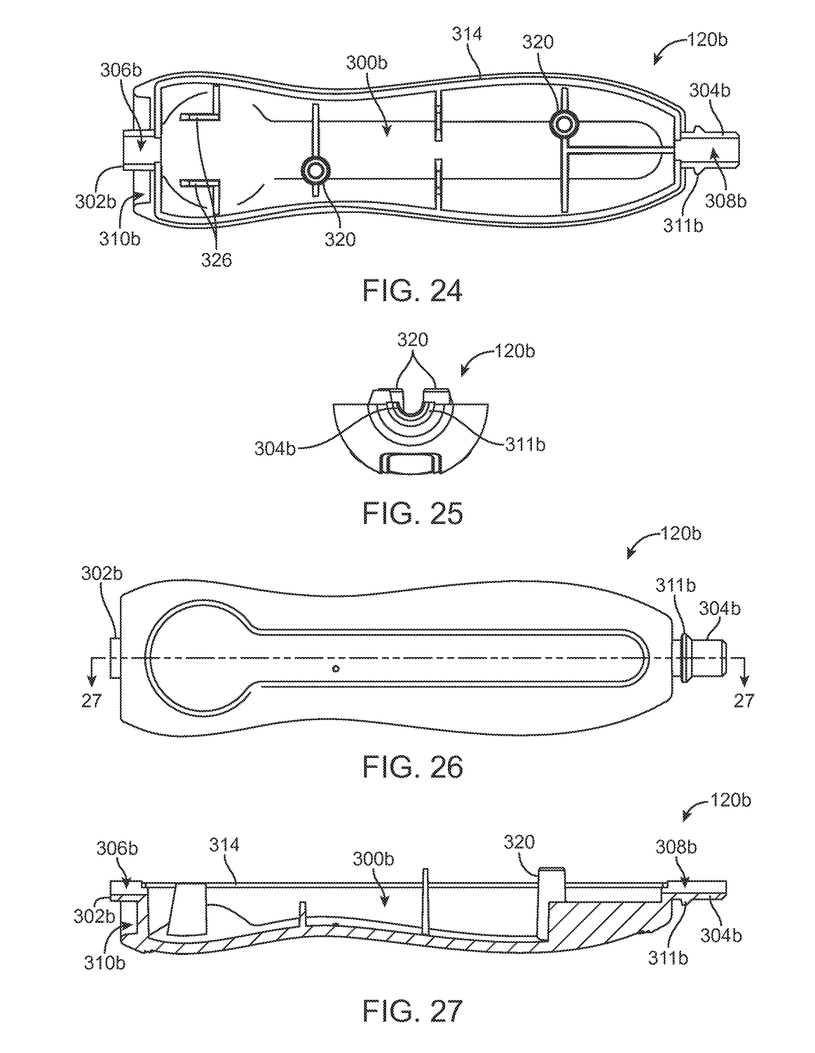

FIG. 19 is an exploded perspective view of the shell 120. FIGS, 20, 21 and 22 are, respectively, a plan view, an end profile view, and a side profile view of the first shell segment 120a. FIG. 23 is a cross-sectional view of the first shell segment 120a taken along a line 23-23 designated in FIG. 22, FIGS. 24, 25, and 26 are, respectively, a plan view, an end profile view, and a side profile view of the second shell segment 120b. FIG. 27 is a cross-sectional view of the second shell segment 120b taken along a line 27-27 designated in FIG. 26. With reference to FIGS. 19-27 together, the first and second shell segments 120a, 120b can be releasably connectable to one another to form the shell 120. The shell 120 can define a cavity 300 and can include distal and proximal collars 302, 304 respectively defining distal and proximal passages 306, 308 opening into the cavity 300. The shell 120 can further define an annular recess 310 extending around the distal collar 302. The annular recess 310 can be shaped to receive a portion of the distal strain-relief element 124. The proximal collar 304 can include an annular proximal flange 311 configured to interact with a distal end of the cable 106 (FIG. 1).

In the illustrated embodiment, the first and second shell segments 120a, 120b form respective halves of the shell 120 and define or include respective halves of certain features of the shell 120. For example, the first and second shell segments 120a, 120b can include respective halves 302a, 302b of the distal collar 302, respective halves 304a, 304b of the proximal collar 304, and respective halves 311a, 311b of the proximal flange 311. The halves 302a, 302b of the distal collar 302 can define the distal passage 306. Similarly, the halves 304a, 304b of the proximal collar 304 can define the proximal passage 308. Furthermore, the first and second shell segments 120a, 120b can define respective halves 300a, 300b of the cavity 300 and respective halves 310a, 310b of the recess 310. In other embodiments, the shell 120 can be unitary, can include more than two releasably connectable segments, or can have another suitable configuration.

With reference again to FIGS. 19-27, the shell 120 can include mating features configured to facilitate coupling the first and second shell segments 120a, 120b to one another. For example, the first shell segment 120a can include a ridge feature 312 extending around its periphery, the second shell segment 120b can include a ledge feature 314 extending around its periphery, and the ledge feature 314 can be configured to snugly receive the ridge feature 312 when the first and second shell segments 120a, 120b are coupled to one another. Similarly, the first shell segment 120a can include cylindrical columns 316 respectively defining hexagonal openings 318, the second shell segment 120b can include cylindrical posts 320, and the columns 316 can be configured to receive the posts 320 within the openings 318 when the first and second shell segments 120a, 120b are coupled to one another. The shell 120 can further include mating features configured to facilitate coupling the shell 120 to the coupler 122 (FIG. 2). For example, at the first shell segment 120a, the shell 120 can include bosses 322 respectively defining bores 324, and, at the second shell segment 120b, the shell 120 can include plates 326.

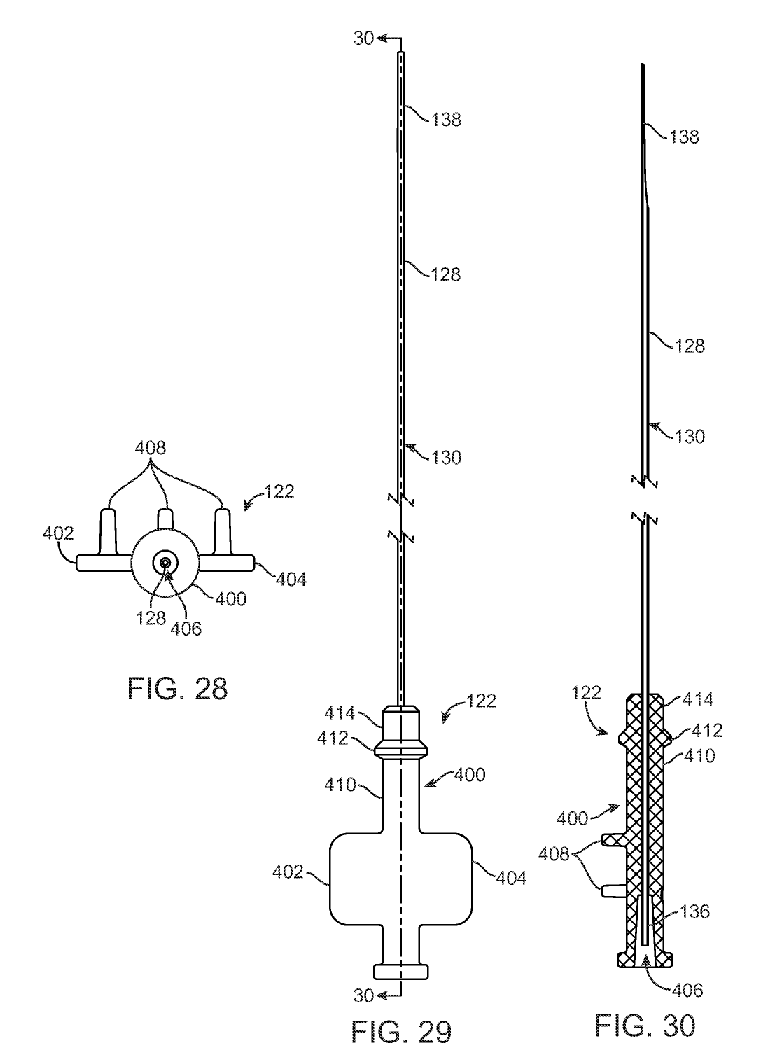

FIGS. 28 and 29 are, respectively, an end profile view and a plan view of the coupler 122 and the proximal hypotube segment 128. FIG. 30 is a cross-sectional view of the coupler 122 and the proximal hypotube segment 128 taken along a line 30-30 designated in FIG. 29. With reference to FIGS. 28-31 together, the coupler 122 can include a cylindrical core 400. The stem 136 of the proximal hypotube segment 128 can be embedded within the core 400. For example, the core 400 can be molded over the stem 136 or connected to the stem 136 in another suitable manner. In this way, the coupler 122 can be fixedly connected to the proximal hypotube segment 128. At its proximal end, the coupler 122 can include a distally tapered channel 406 within which the proximal end of the proximal hypotube segment 128 can be located. The coupler 122 can further include a first wing 402 extending outwardly from the core 400 in a first direction and a second wing 404 extending outwardly from the core 400 in a second direction different than (e.g., circumferentially opposite to) the first direction.

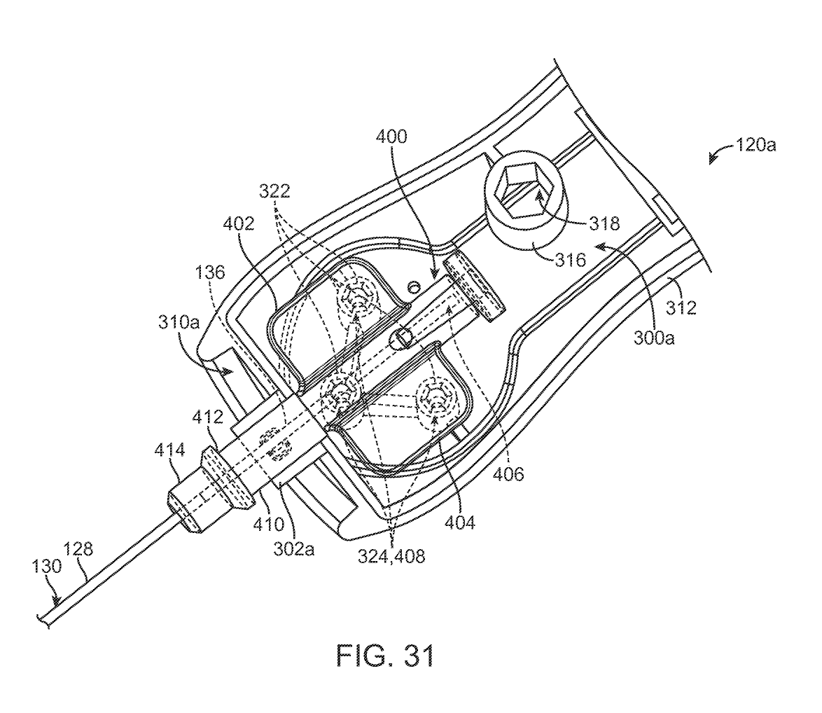

FIG. 31 is a perspective view of the coupler 122, the first shell segment 120a, and the proximal hypotube segment 128 showing the coupler 122 interlockingly connected to the first shell segment 120a. The coupler 122 is transparent in FIG. 31 for purposes of illustration. With reference to FIGS. 19-31 together, the coupler 122 can be interlockingly connected to the handle 110 via the shell 120. For example, the coupler 122 can include mating features corresponding to mating features of the shell 120. In the illustrated embodiment, the coupler 122 includes pegs 408 respectively sized to fit within the bores 324. When the shell 120 and the coupler 122 are operably connected to one another, the bosses 322 can be oriented toward the coupler 122 and the pegs 408 can be oriented toward the shell 120. Individual bosses 322 and corresponding individual pegs 408 can be positioned to engage one another. Contemporaneous engagement of these mating features can at least partially register the coupler 122 relative to the handle 110. When registered, the first and second wings 402, 404 can extend from the core 400 to respective plates 326. In this way, the first and second wings 402, 404 can enhance the positional stability of the coupler 122. Alternatively or in addition, the first and second wings 402, 404 can provide a relatively large area over which the pegs 408 can be distributed. For example, the pegs 408 can be spaced apart from one another in a triangular configuration, with one of the pegs 408 disposed along the first wing 402, another of the pegs 408 disposed along the second wing 404, and another of the pegs 408 disposed along the core 400 offset from a straight line connecting the pegs 408 disposed along the first and second wings 402, 404. This configuration of the pegs 408 is expected to reduce tolerance stack-up and thereby facilitate consistent longitudinal positioning of the proximal hypotube segment 128 relative to the handle 110. In addition or alternatively, the configuration of the pegs 408 can have other advantages.

At successively more distal positions relative to the first and second wings 402, 404, the coupler 122 can include a neck 410, an annular distal flange 412, and a chamfered head 414. When the coupler 122 is operably connected to the handle 110, the core 400, at its neck 410, can extend through the distal passage 306. The distal flange 412 can be distally spaced apart from a distalmost portion of the distal collar 302. The electrode leads and the guide wire can extend through the proximal passage 308, through the cavity 300, into the coupler 122 via the channel 406, into the proximal hypotube segment 128, and extend distally toward the neuromodulation element 112 (FIG, 1).

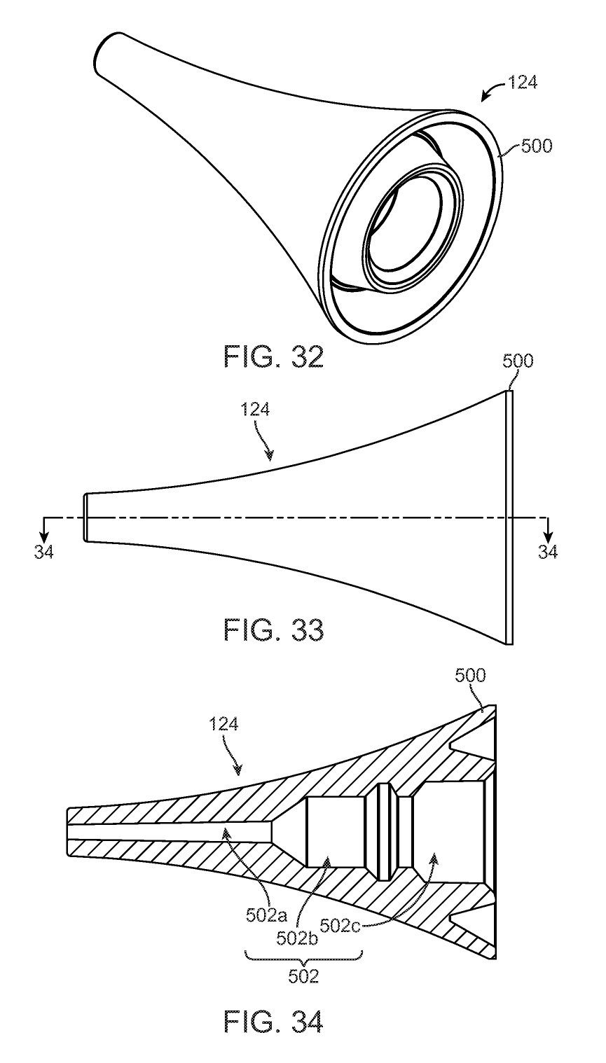

FIGS. 32 and 33 are, respectively, a perspective view and a side profile view of the distal strain-relief element 124. FIG, 34 is a cross-sectional view of the distal strain-relief element 124 taken along a line 34-34 designated in FIG. 33. The distal strain-relief element 124 can be configured to be partially inset into the recess 310 (FIG. 19). For example, the distal strain-relief element 124 can include a rim 500 configured to resiliently deform inwardly within the recess 310 when the distal strain-relief element 124 is urged into engagement with the shell 120 (FIG. 19). The distal strain-relief element 124 can define an interior region 502 having a first portion 502a, a second portion 502b, and a third portion 502c arranged from distal to proximal. The first portion 502a of the interior region 502 can be shaped to snugly receive the proximal hypotube segment 128 so as to reinforce the proximal hypotube segment 128 as it exits the shell 120. The second portion 502b of the interior region 502 can be shaped to snugly receive the head 414 and the distal flange 412. The third portion 502c of the interior region 502 can be shaped to snugly receive the distal collar 302.

When the shell 120, the coupler 122, the proximal hypotube segment 128, and the distal strain-relief element 124 are operably connected to one another, the distal flange 412 can restrict longitudinal displacement of the distal strain-relief element 124 relative to the shell 120 and thereby releasably inhibit separation of the distal strain-relief element 124 from the shell 120. Similarly, the proximal flange 311 can restrict longitudinal displacement of the proximal strain-relief element 125 relative to the shell 120 and thereby releasably inhibit separation of the proximal strain-relief element 125 from the shell 120. When the handle 110 and associated components are assembled, interaction between the distal strain-relief element 124 and the distal collar 302 can releasably inhibit separation of the first and second shell segments 120a, 120b at the distal end of the shell 120. Similarly, interaction between the proximal strain-relief element 125 and the proximal collar 304 can releasably inhibit separation of the first and second shell segments 120a, 120b at the proximal end of the shell 120,

As discussed above, the shaft 108, the neuromodulation element 112, and the coupler 122 can be disposable. Replacing these components can include opening the shell 120 so as to access the coupler 122. Opening the shell 120 can include shifting the distal strain-relief element 124 away from the shell 120 along the shaft 108 and shifting the proximal strain-relief element 125 away from the shell 120 along the cable 106 so as to allow the first and second shell segments 120a, 120b to separate. The distal strain-relief element 124 can be flexible such that the second portion 502b of its interior region 502 stretches in response to firm hand pressure, thereby allowing the distal strain-relief element 12.4 to disengage from the distal flange 412. Similarly, the proximal strain-relief element 125 can be sufficiently flexible to disengage from the proximal flange 311 in response to firm hand pressure. After the shell 120 has been opened, the bosses 322 and the pegs 408 can be separated from one another so that the shaft 108, the neuromodulation element 112, and the coupler 122 can be separated from the shell 120. Next, a new shaft, a new neuromodulation element, and a new coupler can be installed. The first and second shell segments 120a, 120b can then be closed around the new coupler. Finally, the distal and proximal strain-relief elements 124, 125 can be reconnected to the shell 120 to secure the first and second shell segments 120a, 120b to one another. This manner of replacing portions of the catheter 102 is expected to be highly convenient and reliable.

### Renal Neuromodulation

Catheters configured in accordance with at least some embodiments of the present technology can be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, or induced in another suitable manner or combination of manners at one or more suitable treatment locations during a treatment procedure. The treatment location can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery. Various suitable modifications can be made to the catheters described above to accommodate different treatment modalities. For example, the band electrodes 146 (FIG. 2) can be replaced with transducers to facilitate transducer-based treatment modalities.

Renal neuromodulation can include an electrode-based or treatment modality alone or in combination with another treatment modality. Electrode-based or transducer-based treatment can include delivering electricity and/or another form of energy to tissue at or near a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity. A variety of suitable types of energy can be used to stimulate and/or heat tissue at or near a treatment location. For example, neuromodulation in accordance with embodiments of the present technology can include delivering RF energy, pulsed electrical energy, microwave energy, optical energy, focused ultrasound energy (e.g., high-intensity focused ultrasound energy), and/or another suitable type of energy. An electrode or transducer used to deliver this energy can be used alone or with other electrodes or transducers in a multi-electrode or multi-transducer array.

Neuromodulation using focused ultrasound energy (e.g., high-intensity focused ultrasound energy) can be beneficial relative to neuromodulation using other treatment modalities. Focused ultrasound is an example of a transducer-based treatment modality that can be delivered from outside the body. Focused ultrasound treatment can be performed in dose association with imaging (e.g., magnetic resonance, computed tomography, fluoroscopy, ultrasound (e.g., intravascular or intraluminal), optical coherence tomography, or another suitable imaging modality). For example, imaging can be used to identify an anatomical position of a treatment location (e.g., as a set of coordinates relative to a reference point). The coordinates can then entered into a focused ultrasound device configured to change the power, angle, phase, or other suitable parameters to generate an ultrasound focal zone at the location corresponding to the coordinates. The focal zone can be small enough to localize therapeutically-effective heating at the treatment location while partially or filly avoiding potentially harmful disruption of nearby structures. To generate the focal zone, the ultrasound device can be configured to pass ultrasound energy through a lens, and/or the ultrasound energy can be generated by a curved transducer or by multiple transducers in a phased array, which can be curved or straight.

Heating effects of electrode-based or transducer-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° C.) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of luminal structures that perfuse the target neural fibers. In cases where luminal structures are affected, the target neural fibers can be denied perfusion resulting in necrosis of the neural tissue. Heating tissue to a target temperature higher than about 45° C. (e.g., higher than about 60° C.) can induce ablation, fir example, via substantial heating of target neural fibers or of luminal structures that perfuse the target fibers. In some patients, it can be desirable to heat tissue to temperatures that are sufficient to ablate the target neural fibers or the luminal structures, but that are less than about 90° C. (e.g., less than about 85° C., less than about 80° C., or less than about 75° C.).

### ADDITIONAL EXAMPLES

1. A neuromodulation catheter, comprising:
an elongate shaft including—
   a distal end portion,
   a proximal end portion,
   a hypotube segment at a proximal end portion, the hypotube having an outer surface, and
   a jacket disposed around at least a portion of the outer surface, the jacket being made at least partially of a polymer blend including polyether block amide and polysiloxane; and
a neuromodulation element operably connected to the shaft via the distal end portion.

2. The neuromodulation catheter of example 1 wherein the hypotube segment is made at least partially of nickel titanium.

3. The neuromodulation catheter of example 1 or example 2 wherein:
the hypotube segment includes a proximal stem; and
the jacket is not disposed around the outer surface at the proximal stem.

4. The neuromodulation catheter of any of examples 1-3 wherein the jacket is uncoated.

5. The neuromodulation catheter of any of examples 1-4 wherein:
the hypotube segment includes a distal skive; and
the jacket is disposed on at least a portion of the outer surface at the distal skive.

6. The neuromodulation catheter of any of examples 1-5 wherein the jacket includes polysiloxane at greater than 15% by weight.

7. The neuromodulation catheter of any of examples 1-5 wherein the jacket includes polysiloxane at greater than 30% by weight.

8. The neuromodulation catheter of any of examples 1-5 wherein the jacket includes polysiloxane at from 20% to 40% by weight.

9. The neuromodulation catheter of any of examples 1-5 wherein the jacket includes polysiloxane at about 20% by weight and polyether block amide at about 80% by weight.

10. The neuromodulation catheter of any of examples 1-5 wherein the jacket includes polysiloxane at about 40% by weight and polyether block amide at about 60% by weight.

11. A neuromodulation catheter, comprising:
an elongate shaft including
   a distal end portion,
   a proximal end portion, and
   a hypotube segment at the proximal end portion;
a handle operably connected to the shaft via the proximal end portion;
a coupler fixedly connected to the hypotube segment and interlockingly connected to the handle; and
a neuromodulation element operably connected to the shaft via the distal end portion

12. The neuromodulation catheter of example 11 wherein:
the handle includes a shell defining a cavity; and
the coupler is at least partially disposed within the cavity.

13. The neuromodulation catheter of example 12 wherein:
the shell includes a first mating feature oriented toward the coupler;
the coupler includes a second mating feature oriented toward the shell; and
the first and second mating features are positioned to engage one another.

14. The neuromodulation catheter of example 13 wherein:
the shell includes a third mating feature oriented toward the coupler;
the coupler includes a fourth mating feature oriented toward the shell; and
the third and fourth mating features are configured to engage one another.

15. The neuromodulation catheter of example 14 wherein:
the first and third mating features are spaced apart from one another;
the second and fourth mating features are spaced apart from one another; and
contemporaneous engagement of the first and second mating features and of the third and fourth mating features at least partially registers the coupler relative to the handle.

16. The neuromodulation catheter of example 15 wherein:
the first and third mating features individually include a boss defining a bore; and
the second and fourth mating features individually include a peg sized to fit within a corresponding one of the bores.

17. The neuromodulation catheter of example 15 wherein:
the shell includes a fifth mating feature oriented toward the coupler;
the coupler includes a sixth mating feature oriented toward the shell;
the fifth and sixth mating features are configured to engage one another; and
contemporaneous engagement of the first and second mating features, of the third and fourth mating features, and of the fifth and sixth mating features at least partially registers the coupler relative to the handle.

18. The neuromodulation catheter of example 17 wherein:
the hypotube includes a proximal stem;
the coupler includes a core within which the proximal stem is embedded; and
the sixth mating feature is disposed along the core.

19. The neuromodulation catheter of any of examples 12-17 wherein:
the hypotube includes a proximal stem;
the coupler includes a core within which the proximal stem is embedded;
the shell includes a distal collar defining a distal passage opening into the cavity; and
the core extends through the distal passage.

20. The neuromodulation catheter of example 19 wherein:
the coupler includes—
   a first wing extending outwardly from the core in a first direction, and
   a second wing extending outwardly from the core in a second direction different than the first direction;
the second mating feature is disposed along the first wing; and
the fourth mating feature is disposed along the second wing.

21. The neuromodulation catheter of example 20 wherein the first and second directions are circumferentially opposite to one another.

22. The neuromodulation catheter of example 19 wherein:
the coupler includes a flange positioned along the core distally spaced apart from a distalmost portion of the distal collar;
the handle includes a strain-relief element releasably connected to the shell; and
the flange releasably inhibits separation of the strain-relief element from the shell.

23. The neuromodulation catheter of example 22 wherein:
the shell defines an annular recess around the distal collar; and
the strain-relief element is partially inset into the annular recess.

24. The neuromodulation catheter of example 22 wherein:
the shell includes a first segment and a second segment releasably connected to one another; and
the strain-relief element releasably inhibits separation of the first and second segments from one another.

25. The neuromodulation catheter of any of examples 11-24 wherein the hypotube segment is made at least partially of nickel titanium.

26. The neuromodulation catheter of example 25, further comprising a jacket disposed around at least a portion of an outer surface of the hypotube segment.

27. The neuromodulation catheter of example 26 wherein the jacket includes polysiloxane at greater than 15% by weight.

28. The neuromodulation catheter of example 26 wherein the jacket includes polysiloxane at greater than 30% by weight.

29. The neuromodulation catheter of example 26 wherein the jacket includes polysiloxane at from 20% to 40% by weight.

30. The neuromodulation catheter of example 26 wherein the jacket includes polysiloxane at about 20% by weight and polyether block amide at about 80% by weight.

31. The neuromodulation catheter of example 26 wherein the jacket includes polysiloxane at about 40% by weight and polyether block amide at about 60% by weight.

32. A method, comprising:
opening a shell of a handle of a neuromodulation catheter so as to access a first coupler at least partially disposed within the shell, the neuromodulation catheter including an elongate first shaft fixedly connected to the first coupler;
separating two or more spaced-apart mating features of the first coupler from two or more spaced-apart mating features of the shell so as to disengage the first coupler from the handle;
connecting two or more spaced-apart mating features of a second coupler to the two or more spaced-apart mating features of the shell so as to interlockingly connect the second coupler to the handle; and
closing the shell.

33. The method of example 32 wherein opening the shell includes shifting a strain relief element of the handle distally so as to allow first and second segments of the shell to separate.

34. A neuromodulation catheter, comprising:
an elongate shaft; and
a neuromodulation element operably connected to the shaft via a distal end portion of the shaft, the neuromodulation element being movable from a low-profile delivery state to a radially expanded deployed state, the neuromodulation element including—
   a hypotube segment having a first shape when the neuromodulation element is in the delivery state and a second shape when the neuromodulation element is in the deployed state, the second shape being more helical than the first shape,
   a tubular jacket disposed around at least a portion of an outer surface of the hypotube segment, the jacket having reduced-diameter segments spaced apart along its longitudinal axis, and
   band electrodes respectively seated in the reduced-diameter segments and respectively forming closed loops extending circumferentially around the jacket,
wherein a minimum inner diameter of the band electrodes is smaller than a maximum outer diameter of jacket between the reduced-diameter segments.

35. The neuromodulation catheter of example 34 wherein the jacket between the reduced-diameter segments is resilient in response to peristaltic deflection of a magnitude corresponding to a difference between the maximum outer diameter of the jacket between the reduced-diameter segments and the minimum inner diameter of the band electrodes.

36. The neuromodulation catheter of example 34 wherein a maximum outer diameter of the band electrodes and the maximum outer diameter of the jacket between the reduced-diameter segments are at least generally equal.

37. The neuromodulation catheter of any of examples 34-36 wherein the jacket is made at least partially of a polymer blend including polyurethane and polysiloxane.

38. The neuromodulation catheter of any of examples 34-37, further comprising adhesive disposed between the band electrodes and the jacket at the reduced-diameter segments.

39. The neuromodulation catheter of any of examples 34-38 wherein the reduced-diameter segments are fully circumferential.

40. The neuromodulation catheter of any of examples 34-39 wherein:
the jacket includes openings respectively positioned at the reduced-diameter segments;
the neuromodulation catheter further comprises electrical leads respectively connected to the band electrodes; and
the electrical leads respectively extend through the openings.

41. A method for making a neuromodulation element of a neuromodulation catheter, the method comprising:
forming a tubular jacket including a reduced-diameter segment extending through an outer surface of the jacket;
resiliently deforming the jacket inwardly while passing (e.g., advancing or threading) the jacket through a channel of a band electrode so as to move the band electrode toward a longitudinal position at which the band electrode is aligned with the reduced-diameter segment; and
positioning the jacket and a hypotube segment relative to one another so that the jacket is disposed around at least a portion of an outer surface of the hypotube segment.

42. The method of example 41 wherein:
the reduced-diameter segment, the band electrode, and the longitudinal position are a first reduced-diameter segment, a first band electrode, and a first longitudinal position respectively;
forming the jacket includes funning the jacket to include a second reduced-diameter segment extending through the outer surface of the jacket, the first and second reduced-diameter segments being spaced apart along a longitudinal axis of the jacket; and
the method further comprises resiliently deforming the jacket inwardly while passing the jacket through a channel of a second band electrode so as to move the second band electrode toward a second longitudinal position at which the second band electrode is aligned with the second reduced-diameter segment.

43. The method of example 41 or example 42 wherein the method does not include swaging the jacket after forming the jacket and before positioning the jacket and the hypotube segment relative to one another.

44. The method of any of examples 41-43 wherein forming the jacket includes forming the jacket by injection molding.

45. The method of any of examples 41-43 wherein forming the jacket includes:
forming a tubular blank by extrusion; and
removing a portion of the blank to form the reduced-diameter segment.

46. The method of example 45 wherein removing the portion of the blank includes removing the portion of the blank by laser ablation.

47. A neuromodulation catheter, comprising:
an elongate shaft including—
   a distal end portion,
   a proximal end portion,
   a hypotube segment at a proximal end portion, the hypotube having an outer surface, and
   a jacket disposed around at least a portion of the outer surface, the jacket being made at least partially of a polymer blend including polyether block amide and polysiloxane; and
a neuromodulation element operably connected to the shaft via the distal end portion, wherein the jacket includes polysiloxane at greater than 30% by weight.

48. The neuromodulation catheter of example 47 wherein the hypotube segment is made at least partially of nickel titanium.

49. The neuromodulation catheter of example 47 or example 48 wherein:
the hypotube segment includes a proximal stem; and
the jacket is not disposed around the outer surface at the proximal stem.

50. The neuromodulation catheter of any of examples 47-49 wherein the jacket is uncoated.

51. The neuromodulation catheter of any of examples 47-50 wherein:
the hypotube segment includes a distal skive; and
the jacket is disposed on at least a portion of the outer surface at the distal skive.

52. The neuromodulation catheter of any of examples 47-51 wherein the jacket includes polysiloxane at about 40% by weight and polyether block amide at about 60% by weight.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown and/or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may have been disclosed in the context of those embodiments, other embodiments may also exhibit such advantages, and not all embodiments need necessarily exhibit such advantages or other advantages disclosed herein to fall within the scope of the present technology. Accordingly, this disclosure and associated technology can encompass other embodiments not expressly shown and/or described herein.

The methods disclosed herein include and encompass, in addition to methods of practicing the present technology (e.g., methods of making and using the disclosed devices and systems), methods of instructing others to practice the present technology. For example, a method in accordance with a particular embodiment includes forming a tubular jacket, resiliently deforming the jacket inwardly while passing the jacket through a channel of a band electrode, and positioning the jacket and a hypotube segment relative to one another so that the jacket is disposed around at least a portion of an outer surface of the hypotube segment. A method in accordance with another embodiment includes instructing such a method.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments of the present technology.

I/We claim:

1. A neuromodulation catheter, comprising:
an elongate shaft including—
a distal end portion,
a proximal end portion, and
a hypotube segment at the proximal end portion;
a handle operably connected to the shaft via the proximal end portion;
a coupler fixedly connected to the hypotube segment and interlockingly connected to the handle; and
a neuromodulation element operably connected to the shaft via the distal end portion.

2. The neuromodulation catheter of claim 1 wherein:
the handle includes a shell defining a cavity; and
the coupler is at least partially disposed within the cavity.

3. The neuromodulation catheter of claim 2 wherein:
the shell includes a first mating feature oriented toward the coupler;
the coupler includes a second mating feature oriented toward the shell; and
the first and second mating features are positioned to engage one another.

4. The neuromodulation catheter of claim 3 wherein:
the shell includes a third mating feature oriented toward the coupler;
the coupler includes a fourth mating feature oriented toward the shell; and
the third and fourth mating features are configured to engage one another.

5. The neuromodulation catheter of claim 4 wherein:
the first and third mating features are spaced apart from one another;
the second and fourth mating features are spaced apart from one another; and
contemporaneous engagement of the first and second mating features and of the third and fourth mating features at least partially registers the coupler relative to the handle.

6. The neuromodulation catheter of claim 5 wherein:
the first and third mating features individually include a boss defining a bore; and
the second and fourth mating features individually include a peg sized to fit within a corresponding one of the bores.

7. The neuromodulation catheter of claim 5 wherein:
the shell includes a fifth mating feature oriented toward the coupler;
the coupler includes a sixth mating feature oriented toward the shell;
the fifth and sixth mating features are configured to engage one another; and
contemporaneous engagement of the first and second mating features, of the third and fourth mating features, and of the fifth and sixth mating features at least partially registers the coupler relative to the handle.

8. The neuromodulation catheter of claim 7 wherein:
the hypotube includes a proximal stem;
the coupler includes a core within which the proximal stem is embedded; and
the sixth mating feature is disposed along the core.

9. The neuromodulation catheter of claim 2 wherein:
the hypotube includes a proximal stem;
the coupler includes a core within which the proximal stem is embedded;
the shell includes a distal collar defining a distal passage opening into the cavity; and
the core extends through the distal passage.

10. The neuromodulation catheter of claim 9 wherein:
the coupler includes—
a first wing extending outwardly from the core in a first direction, and
a second wing extending outwardly from the core in a second direction different than the first direction;
the second mating feature is disposed along the first wing; and
the fourth mating feature is disposed along the second wing.

11. The neuromodulation catheter of claim 10 wherein the first and second directions are circumferentially opposite to one another.

12. The neuromodulation catheter of claim 9 wherein:
the coupler includes a flange positioned along the core distally spaced apart from a distalmost portion of the distal collar;
the handle includes a strain-relief element releasably connected to the shell; and
the flange releasably inhibits separation of the strain-relief element from the shell.

13. The neuromodulation catheter of claim 12 wherein:
the shell defines an annular recess around the distal collar; and
the strain-relief element is partially inset into the annular recess.

14. The neuromodulation catheter of claim 12 wherein:
the shell includes a first segment and a second segment releasably connected to one another; and
the strain-relief element releasably inhibits separation of the first and second segments from one another.

15. The neuromodulation catheter of claim 1 wherein the hypotube segment is made at least partially of nickel titanium.

16. The neuromodulation catheter of claim 1, further comprising a jacket disposed around at least a portion of an outer surface of the hypotube segment.

17. The neuromodulation catheter of claim 16 wherein the jacket includes polysiloxane at greater than 15% by weight.

18. The neuromodulation catheter of claim 16 wherein the jacket includes polysiloxane at greater than 30% by weight.

19. The neuromodulation catheter of claim 16 wherein the jacket includes polysiloxane at from 20% to 40% by weight.

20. The neuromodulation catheter of claim 16 wherein the jacket includes polysiloxane at about 20% by weight and polyether block amide at about 80% by weight.

21. The neuromodulation catheter of claim 16 wherein the jacket includes polysiloxane at about 40% by weight and polyether block amide at about 60% by weight.

22. A neuromodulation catheter, comprising:
an elongate shaft including—
a distal end portion,
a proximal end portion,
a hypotube segment at a proximal end portion, the hypotube having an outer surface, and
a jacket disposed around at least a portion of the outer surface, the jacket being made at least partially of a polymer blend including polyether block amide and polysiloxane; and
a neuromodulation element operably connected to the shaft via the distal end portion, wherein the jacket includes polysiloxane at greater than 30% by weight.

23. The neuromodulation catheter of claim 22 wherein the hypotube segment is made at least partially of nickel titanium.

24. The neuromodulation catheter of claim 22 wherein:
the hypotube segment includes a proximal stem; and
the jacket is not disposed around the outer surface at the proximal stem.

25. The neuromodulation catheter of claim 22 wherein the jacket is uncoated.

26. The neuromodulation catheter of claim 22 wherein:
the hypotube segment includes a distal skive; and
the jacket is disposed on at least a portion of the outer surface at the distal skive.

27. The neuromodulation catheter of claim 22 wherein the jacket includes polysiloxane at about 40% by weight and polyether block amide at about 60% by weight.
